(19) United States
(12) Patent Application Publication
(10) Pub. No.: US 2026/0250659 A1
(43) Pub. Date: Aug. 27, 2026

(54) METHOD AND SYSTEMS FOR ISOLATING NUCLEAR RNA FROM A HIGH DEGRADATION TISSUE

(71) Applicant: Case Western Reserve University, Cleveland, OH (US)

(72) Inventors: Paul J. Tesar, Wickliffe, OH (US); Marissa Scavuzzo, Cleveland, OH (US)

(21) Appl. No.: 19/489,730

(22) PCT Filed: Jun. 6, 2024
(86) PCT No.: PCT/US2024/032676

§ 371 Date: Dec. 3, 2025

Related U.S. Application Data
(60) Provisional application No. 63/506,710, filed on Jun. 7, 2023.

(51) Int. Cl. C12N 15/10; C12Q 1/6806; C12Q 1/6869

(52) U.S. Cl. CPC C12N 15/1017; C12Q 1/6806; C12Q 1/6869

(57) ABSTRACT

The invention described herein provides an improved method and reagents to isolate nuclear RNA from high degradation tissues, such as duodenum, for use in, for example, high resolution transcriptome profiling and analysis.

### RELATED APPLICATION

The instant application claims priority to U.S. Provisional Application No. 63/506,710, filed Jun. 7, 2023, the entire contents of which are expressly incorporated by reference herein.

### GOVERNMENT SUPPORT

This invention was made with government support under Grant No. R35NS116842, awarded by the National Institute of Health. The government has certain rights in the invention.

### BACKGROUND OF THE INVENTION

In many organs, single cell technologies, including single nucleus RNA-sequencing (snRNA-seq), have increased our understanding of cellular diversity in complex tissues and enabled the identification of specialized cell types with unique roles in tissue function. Single nucleus RNA-sequencing allows for transcriptomic analysis regardless of cell durability. However, many tissues are highly degradative in nature due to their inherent biology, and many tissue samples derived from surgical resection or autopsy have unstable RNAs. For example, the most proximal region of the small intestine, the duodenum, is the epicenter of digestion, and houses an abundance of RNAses, enzymes, and ions. These technical challenges have prevented transcriptional analysis of the adult whole duodenum at the single cell level, with previous studies restricted to embryonic or early postnatal stages or relying on sorting for marked nuclei. These obstacles have hindered our ability to disentangle the repertoire of cell types and their impact on surrounding cells at stages when the duodenum is fully functional.

### SUMMARY OF THE INVENTION

One aspect of the invention provides a method for isolating nuclear RNA from a high degradation tissue, said method comprising: i) incubating on ice the high degradation tissue reduced to a suitable size in a lysis buffer, for about 5-9 minutes (e.g., about 6-8 minutes, about 6.5-7.5 minutes, or about 7 minutes), wherein the lysis buffer comprises RNase inhibitor, protease inhibitor, buffer, osmotic stabilizer and ions to maintain/stabilize organelle membrane integrity, DNA precipitation reagent, chelating agent that quenches free Mg²⁺, and/or optionally anti-oxidant or reducing agent; ii) homogenizing the high degradation tissue of the suitable size in the lysis buffer through sheer mechanical stress to generate a loosely homogenized tissue, wherein the sheer mechanical stress is generated by a Dounce-type homogenizer having a loose or Type-A pestle, through a total of about 15-25 passes (e.g., about 20 passes) of the Type-A pestle in the Dounce-type homogenizer, wherein the outer diameter of the Type-A homogenizer has a clearance from the inner diameter of the cylinder wall of between about 0.0025-0.0055 inches; iii) adding a detergent mix, at about 6-10% (e.g., about 8%) volume of the lysis buffer, to the loosely homogenized tissue, wherein the detergent mix comprises about 4-6% or about 5% of IGEPAL CA-630-type nonionic, non-denaturing detergent in the lysis buffer; iv) homogenizing the loosely homogenized tissue with the detergent mix in order to release organelles into said lysis buffer, using a tight or Type-B pestle in the Dounce-type homogenizer, by about 35-45 passes (e.g., about 40 passes) of the Type-B pestle in the Dounce-type homogenizer, wherein the outer diameter of the Type-B homogenizer has a clearance from the inner diameter of the cylinder wall of between about 0.0005-0.0025 inches; v) filtering tissue lysates generated by step iv) through a strainer having a sieve size of about 40 μm to collect nuclei in the pass-through portion; vi) adding an equal volume of a 50% iodixanol solution to the pass-through portion and vortexing to create a mixture having about 25% iodixanol; vii) underlaying the mixture with an about 30% iodixanol solution and an about 40% iodixanol solution without disturbing the resulting three-layer iodixanol gradient; viii) centrifuging the three-layer iodixanol gradient at about 10,000 g for 15-25 minutes (e.g., about 18 minutes) at about 4° C. with no brake; ix) collecting nuclei from the interface(s) between the layers of the iodixanol gradient into a nuclei wash buffer for washing through vortexing, up to twice; and, x) filtering washed nuclei through a 20 μm filter before pelleting the nuclei by centrifugation at about 500 g (e.g., for about 5 minutes at about 4° C.).

Another aspect of the invention comprises a method of profiling transcriptome based on single nucleus RNA-sequencing (snRNA-seq), the method comprising: 1) isolating nuclear RNA using the method of the invention; and, 2) profiling transcriptome of the isolated nuclear RNA using single nucleus RNA-sequencing (snRNA-seq).

### BRIEF DESCRIPTION OF THE DRAWINGS

FIGS. 1A-1D show nuclei isolation from high degradation tissues using established protocols. FIG. 1A shows brightfield images of isolated nuclei stained with 0.4% Trypan blue using three established methods (A) (Hill and Martin, Epigenetic Assays in Purified Cardiomyocyte Nuclei. Methods Mol Biol 2158, 307-321 (2021)), (B) (Drokhlyansky et al., The Human and Mouse Enteric Nervous System at Single-Cell Resolution. Cell 182, 1606-1622 e1623 (2020)), and (C) (Tosti et al., Single-Nucleus and In Situ RNA-Sequencing Reveal Cell Topographies in the Human Pancreas. Gastroenterology 160, 1330-1344 e1311 (2021)), and an exemplary embodiment of the invention (e.g., “CitraPrep”). FIG. 1B is the image of an RNA formaldehyde gel showing ribosomal RNA 28S and 18S bands in bulk postnatal day 21 duodenal tissue isolated immediately by acid-guanidinium-phenol based extraction compared to nuclei preparations from the cortex and the heart using an established method. FIG. 1C is the image of an RNA formaldehyde gel showing ribosomal RNA 28S and 18S bands in bulk postnatal day 21 (PN21) duodenal tissue isolated immediately by acid-guanidinium-phenol based extraction compared to nuclei preparations from the duodenum using 3 different established methods (A)(supra), (B)(supra), and (C)(supra). FIG. 1D is the image of an RNA formaldehyde gel showing ribosomal RNA 28S and 18S bands in bulk PN1 and PN21 duodenal tissue isolated immediately by acid-guanidinium-phenol based extraction compared to snap frozen tissue that incubated undisturbed for 1 hour (1 hr) on ice prior to isolation using acid-guanidinium-phenol extraction.

FIG. 2 shows optimization of CitraPrep for single nucleus RNA-seq of high degradation tissue. RNA formaldehyde gel showing ribosomal RNA 28S and 18S bands in bulk PN1 and PN21 duodenal tissue isolated immediately by acid-guanidinium-phenol based extraction compared to iterations of nuclei preparation chemistry. Ratio of 28S to 18S band is shown below to score each method. The 10 mM citric acid, 7 minute incubation time resulted in the best results by 28S:18S ratio at 87.1.

FIGS. 3A-3B show that CitraPrep produces high quality RNA from individual nuclei. FIG. 3A shows percent RNA concentration measured in supernatant after final centrifugation in nuclei preparation protocol in relation to nuclear RNA content compared to an established nuclear RNA protocol. FIG. 3B shows percent mitochondrial reads sequenced in each individual animal after running CellBender but prior to any preprocessing or filtering showing low cytoplasmic contamination. Each dot is an individual nuclear transcriptome with percent mt-transcripts (mitochondrial transcripts) on the y-axis. Cutoffs for filtering for further analysis were set at 5% and shown by a dashed red line.

FIGS. 4A-4E show blueprints of the whole adult mouse duodenum. FIG. 4A shows labeling of the enteric nervous system in the adult Sox10-cre; Ai14(RCL-tdT) mouse duodenum. RFP marks enteric nervous system cells, GFAP is shown in green, and nuclei are marked in white by DAPI. Scale bar=100 μm. FIG. 4B shows the proportion of cell types in the adult mouse duodenum from CitraPrep and snRNA-seq (n=4 mice, 57,033 nuclei). FIG. 4C shows snRNAseq of duodenal nuclei visualized by Uniform Manifold Approximation and Projection (UMAP), colored by cell type identity, and annotated post hoc. FIG. 4D shows proportion of broad cell types captured in each of our biological replicates. FIG. 4E shows cell type-specific marker expression (columns) in clusters (rows) as shown in FIG. 4C. Broad cell type classification is shown with dark gray boxes indicating from left to right epithelial cells, mesenchymal cells, immune cells, vasculature, musculature, and enteric nervous system cells. The size of each circle indicates percentage of cells in the cluster that express the marker (>1 UMI) while the color shows the average expression of transcript in cells. ISC, intestinal stem cell; EEC, enteroendocrine cell; CHE, Cftr high expressing epithelial cell; lymph, lymphocytes; ICC, interstitial cells of Cajal; ENS, enteric nervous system.

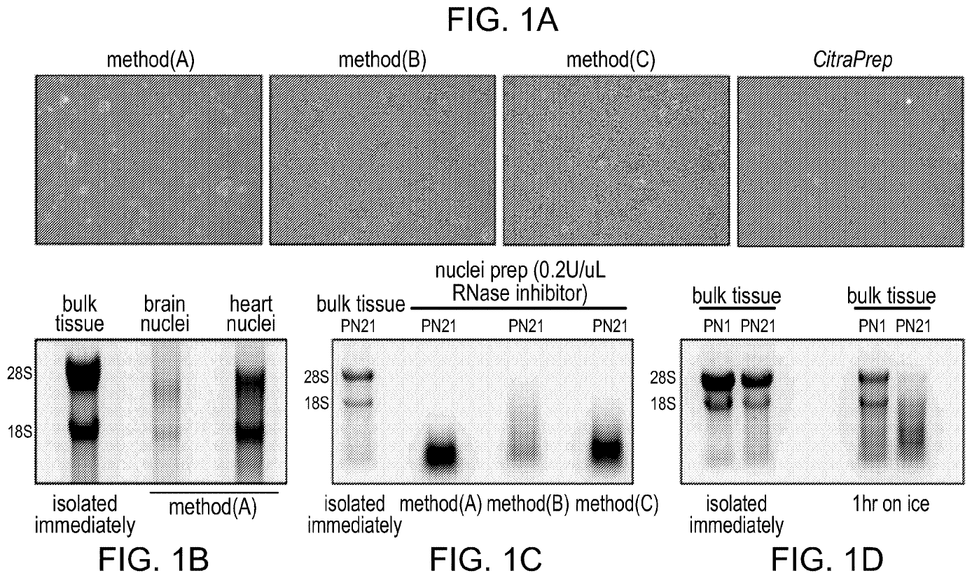

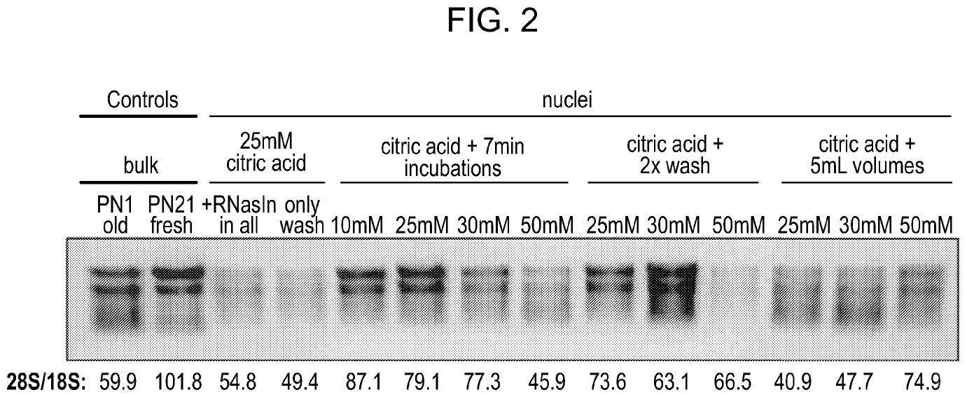

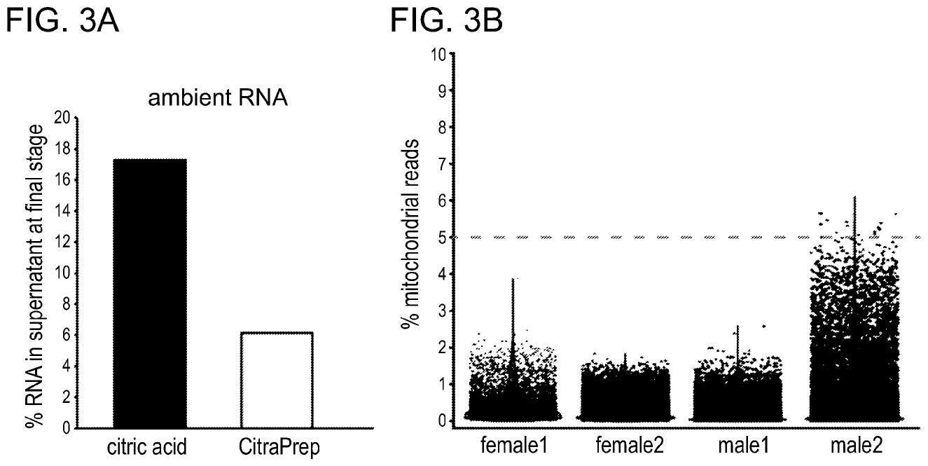

### DETAILED DESCRIPTION OF THE INVENTION

Described herein is a new technology that overcomes the technical limitations that have to this date prevented global analysis of the adult duodenum at the single cell level.

Based on testing over numerous permutations and combinations of conditions, including a dozen iterations of extraction methods (dounced, crushed, homogenized), incubation times (5 minutes and 7 minutes), washing conditions (single, double, or triple washed), wash volumes (5 mL, 10 mL, and 50 mL), chelating agent concentrations to quench free Mg²⁺ ions (10 mM, 25 mM, 30 mM, and 50 mM citric acid), RNAse inhibitor concentrations (0.2 U/μL, 0.5 U/μL, and 1 U/μL), and RNAse inhibitor enzyme mixes (SUPERase, RNasin, Protector, and in varying combination), Applicant has identified the combination of specific methodological approaches and chemical composition for successful profiling.

More specifically, Applicant scored each condition by 28S:18S ribosomal RNA band ratios and percent ambient RNA, naming the top condition “CitraPrep”—a specific embodiment of the invention described herein.

The new methods described herein for nuclei isolation has allowed for the first high quality, low contamination molecular maps of the proximal small intestine, and can be applied to a wide range of tissues.

Potential use of this technology includes single nucleus RNA-sequencing from tissues that are otherwise difficult to profile. This includes the duodenum, ileum, colon, pancreas, and human tissue samples. Several companies develop and sell nuclei isolation kits that are optimized for specific tissues (for example, 10X Genomics: pages.10xgenomics.com/rs/446-PBO-704/images/10x_LIT000163_Product_Sheet_Nuclei_Isolation_Kit_Letter_digital.pdf). The development of CitraPrep provides a useful approach to expand the tissues that are able to be profiled. This technology has the ability to revolutionize our ability to profile high degradation tissues.

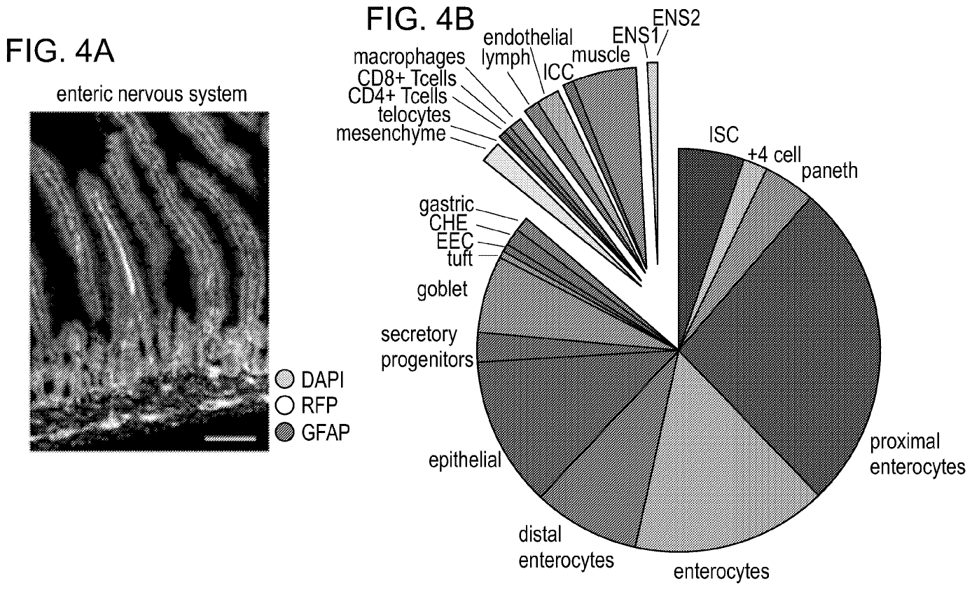

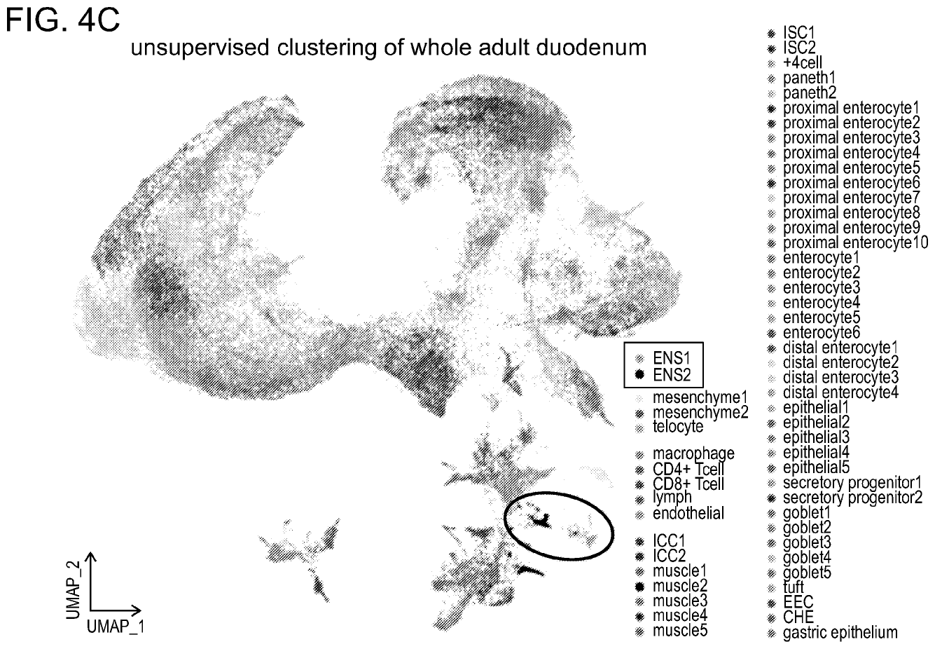

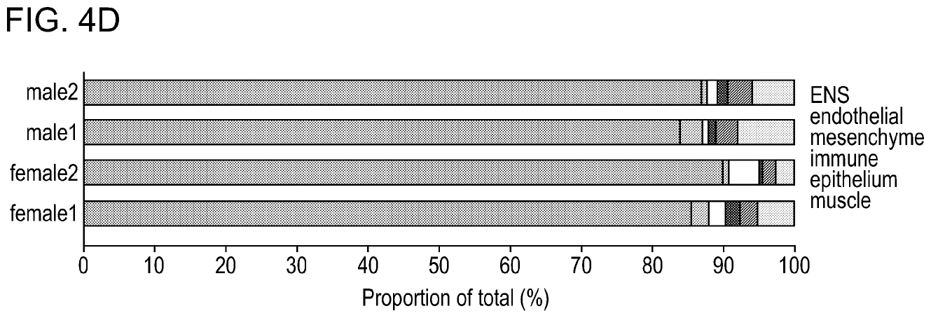

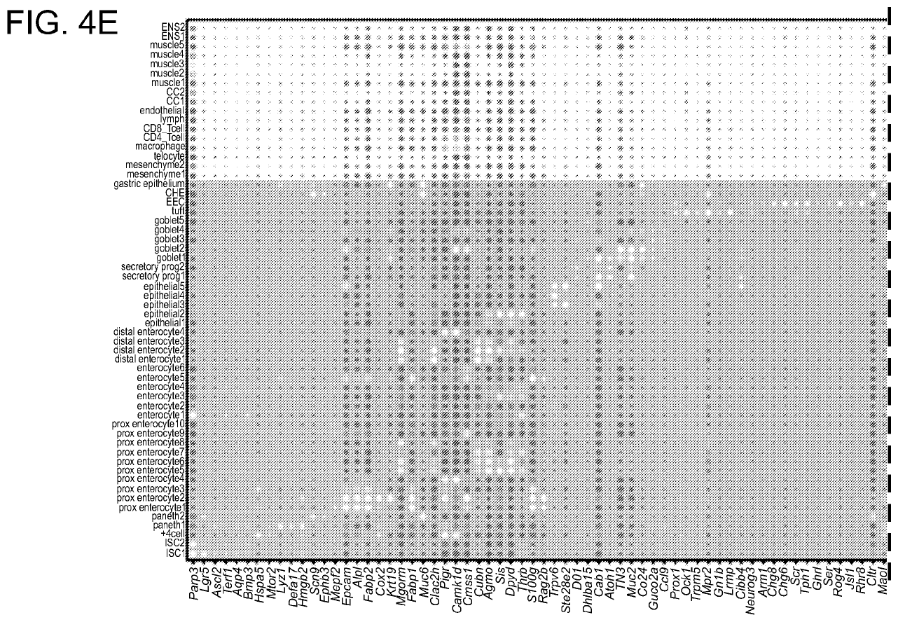

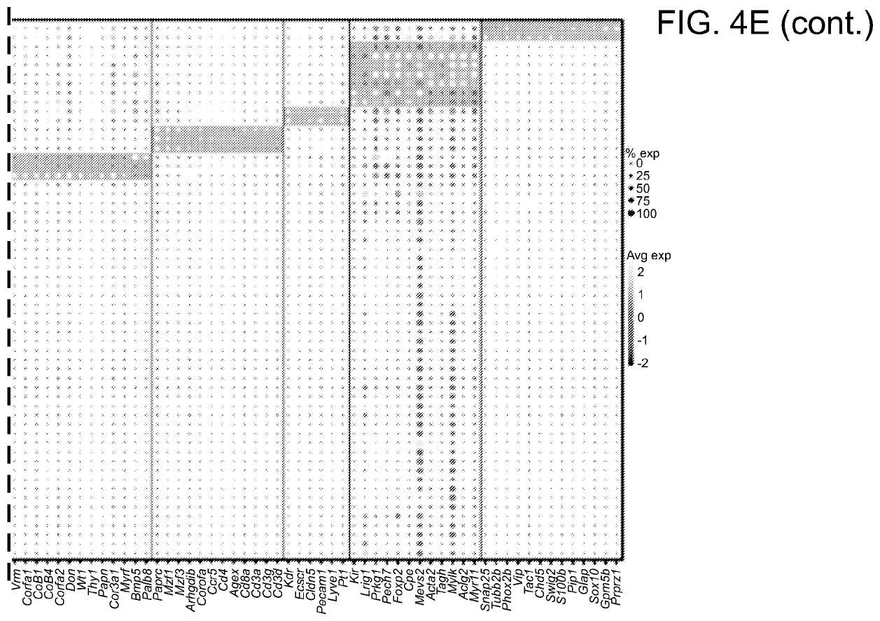

An exemplary use of this technology is the generation of molecular blueprints of the adult mouse duodenum (see FIG. 4). No global maps have been able to capture the repertoire of cell types throughout the adult duodenum due to the diverse cell types and high level of RNAses and ions. For example, the enteric nervous system is fragile and embedded in every layer of the gut, within the rigid muscle layers and throughout the delicate mucosa, making dissociating tissue for single cell profiling impossible without introducing biases in cell populations. Our example shows how using CitraPrep allows for the unbiased identification of enteric nervous system cells throughout the intestine.

Thus in one aspect, the invention provides a method for isolating nuclear RNA from a high degradation tissue, said method comprising: i) incubating on ice the high degradation tissue reduced to a suitable size in a lysis buffer, for about 5-9 minutes (e.g., about 6-8 minutes, about 6.5-7.5 minutes, or about 7 minutes), wherein the lysis buffer comprises RNase inhibitor, protease inhibitor, buffer, osmotic stabilizer and ions to maintain/stabilize organelle membrane integrity, DNA precipitation reagent, chelating agent that quenches free Mg²⁺, and/or optionally anti-oxidant or reducing agent; ii) homogenizing the high degradation tissue of the suitable size in the lysis buffer through sheer mechanical stress to generate a loosely homogenized tissue, wherein the sheer mechanical stress is generated by a Dounce-type homogenizer having a loose or Type-A pestle, through a total of about 15-25 passes (e.g., about 20 passes) of the Type-A pestle in the Dounce-type homogenizer, wherein the outer diameter of the Type-A homogenizer has a clearance from the inner diameter of the cylinder wall of between about 0.0025-0.0055 inches; iii) adding a detergent mix, at about 6-10% (e.g., about 8%) volume of the lysis buffer, to the loosely homogenized tissue, wherein the detergent mix comprises about 4-6% or about 5% of IGEPAL CA-630-type nonionic, non-denaturing detergent in the lysis buffer; iv) homogenizing the loosely homogenized tissue with the detergent mix in order to release organelles into said lysis buffer, using a tight or Type-B pestle in the Dounce-type homogenizer, by about 35-45 passes (e.g., about 40 passes) of the Type-B pestle in the Dounce-type homogenizer, wherein the outer diameter of the Type-B homogenizer has a clearance from the inner diameter of the cylinder wall of between about 0.0005-0.0025 inches; v) filtering tissue lysates generated by step iv) through a strainer having a sieve size of about 40 μm to collect nuclei in the pass-through portion; vi) adding an equal volume of a 50% iodixanol solution to the pass-through portion and vortexing to create a mixture having about 25% iodixanol; vii) underlaying the mixture with an about 30% iodixanol solution and an about 40% iodixanol solution without disturbing the resulting three-layer iodixanol gradient; viii) centrifuging the three-layer iodixanol gradient at about 10,000 g for 15-25 minutes (e.g., about 18 minutes) at about 4° C. with no brake; ix) collecting nuclei from the interface(s) between the layers of the iodixanol gradient into a nuclei wash buffer for washing through vortexing, up to twice; and, x) filtering washed nuclei through a 20 μm filter before pelleting the nuclei by centrifugation at about 500 g (e.g., for about 5 minutes at about 4° C.).

In certain embodiments, the high degradation tissue derives from a surgical resection or autopsy, or is rich in RNAses, proteases, enzymes, and/or ions (e.g., an enteric tissue, such as the most proximal region of the small intestine, duodenum, ileum, colon, or pancreas).

In certain embodiments, the suitable size is no more than about 3 mm, 2 mm, or 1 mm in any dimension.

In certain embodiments, step i) is about 7 minutes on ice, optionally with about 4 mL of the lysis buffer for a total tissue amount of less than about 15 mm in any dimension.

In certain embodiments, the lysis buffer comprises: a) an RNase inhibitor selected from the group consisting of RNasin (e.g., about 0.25-1 U/μL, or about 0.5 U/μL of RNasin) and SUPERase RNAse Inhibitor (e.g., about 0.25-1 U/μL, or about 0.5 U/μL of SUPERase RNAse Inhibitor); b) a protease inhibitor (e.g., complete, EDTA-free protease inhibitor); c) a buffer that maintains pH at about 7.5-8 (e.g., about 20 mM Tricine-KOH, pH 7.8); d) a DNA precipitation reagent selected from the group consisting of spermine tetrahydrochloride (e.g., about 0.15 mM spermine tetrahydrochloride) and spermidine trihydrochloride (e.g., about 0.5 mM spermidine trihydrochloride); e) an osmotic stabilizer (such as 0.25 M sucrose); f) ions to maintain/stabilize organelle membrane integrity (such as about 20-30 mM or about 25 mM KCl, and about 2.5-7.5 mM or about 5 mM MgCl₂); g) a chelating agent to quench free Mg²⁺(such as about 5-20 mM or about 10 mM citric acid); and, h) an anti-oxidant or reducing agent (such as about 1 mM DTT).

In certain embodiments, the IGEPAL CA-630-type nonionic, non-denaturing detergent comprises IGEPAL CA-630 (octylphenoxypolyethoxyethanol) or Nonidet P-40 (NP-40).

In certain embodiments, the concentrations of the components of the lysis buffer remain substantially unchanged through steps i)-viii).

In certain embodiments, step ix) is performed in about 10 mL of the nuclei wash buffer.

In certain embodiments, step ix) consists of two washes, each in about 10 mL of the nuclei wash buffer.

In certain embodiments, the method further comprises resuspending collected nuclei in the nuclei wash buffer for counting (e.g., in hemocytometer) and/or observation for quality control.

In certain embodiments, the nuclei wash buffer comprises 1% serum albumin (such as BSA), an RNase inhibitor (such as RNasin (e.g., about 0.25-1 U/μL, or about 0.5 U/μL of RNasin) and/or SUPERase RNAse Inhibitor (e.g., about 0.25-1 U/μL, or about 0.5 U/μL of SUPERase RNAse Inhibitor)) in a buffer (such as PBS).

In certain embodiments, nuclear RNA isolated by the method has an 28S RNA:18S RNA ratio of at least about 50, 55, 60, 65, 70, 75, 80, 85 or more.

Another aspect of the invention comprises a method of profiling transcriptome based on single nucleus RNA-sequencing (snRNA-seq), the method comprising: 1) isolating nuclear RNA using the method of the invention; and, 2) profiling transcriptome of the isolated nuclear RNA using single nucleus RNA-sequencing (snRNA-seq).

In certain embodiments, the nuclear RNA is isolated from a high degradation tissue that either derives from a surgical resection or autopsy, or is rich in RNAses, proteases, enzymes, and/or ions (e.g., an enteric tissue, such as the most proximal region of the small intestine, duodenum, ileum, colon, or pancreas).

It should be understood that any one embodiment described herein, including those described only in the Example or claims, can be combined with any one or more additional embodiments of the invention unless expressly disclaimed or being improper.

### EXAMPLES

The following is an illustrative embodiment to highlight certain non-limiting features of the invention described herein, and thus should not be construed to limit the scope of the invention described herein in any respect.

### Example 1 Citraprep: a Nuclear RNA Isolation Method for High Degradation Tissues

Snap frozen tissues were stored at −80° C. until nuclei isolation. Glass dounce homogenizers were placed on ice (Wheaton Science Products, 357542). Frozen tissue was placed into a petri dish on ice and cut into 0.2 cm cubed pieces or smaller using clean scissors and forceps in 1 mL of CitraHB. Chopped tissue (totaling less than 1.5 cm cubed) was transferred to the glass dounce homogenizer on ice, and 4 mL of CitraHB was added. Tissue was incubated on ice for 7 minutes. Tissue was homogenized 20× with loose pestle before adding 320 μL of detergent mix, and homogenizing 40× with tight pestle. If tissue did not homogenize, an electric homogenizer with short bursts was used on ice. Nuclei were observed under the microscope for quality control before proceeding.

Tissue lysate were filtered through a 40 μm strainer into a 50 mL conical tube. Next, 5 mL of 50% iodixanol solution was added to the filtered tissue lysate to make a 25% iodixanol solution and vortexed to mix. The tissue lysate was slowly underlaid with 7.5 mL of 30% iodixanol solution, then slowly underlaid with 4 mL of 40% iodixanol solution. Without disturbing the layers, the 50 mL tube was weighed and balanced before centrifugation at 10,000 g for 18 minutes at 4° C. with no brake.

Nuclei were collected from the interface layer with a 1000 mL pipette and placed into a new tube with 10 mL nuclei wash buffer and vortexed, before filtering through a 20 μm filter. Nuclei were pelleted by centrifugation at 500 g for 5 minutes at 4° C. Supernatant were removed, and nuclei were washed a second time. The pellet was resuspended in 250 μL of nuclei wash buffer and nuclei are counted using a hemocytometer before proceeding with single cell applications.

The various solutions used in the example about were listed below. It should be noted that the specific concentrations of ingredients in each of the solutions are merely for illustrative purpose only, and are not in any way limiting. One of skill in the art would readily understand that minor adjustments based on specific experimental materials and conditions etc. can be achieved without departing from the spirit of the invention.

Lysis Buffer (e.g., CitraHB) 0.25 M sucrose; 25 mM KCl; 5 mM MgCl₂; 20 mM Tricine-KOH pH 7.8; 10 mM citric acid; 0.5 U/μL RNasin (Promega, N2615); 0.5 U/μL SUPERase RNase inhibitor (Thermo Fisher, AM2696); 1 mM DTT; 0.15 mM spermine tetrahydrochloride; 0.5 mM spermidine trihydrochloride; cOmplete™, EDTA-free protease inhibitor (Sigma, 11836170001).

### Detergent Mix

50 μL Igepal CA-630; 950 μL CitraHB.

### Diluent

150 mM KCl; 30 mM MgCl₂; 120 mM Tricine-KOH pH 7.8; and 10 mM citric acid.

### 50% Iodixanol

5 volumes OptiPrep; 1 volume of Diluent; 0.5 U/μL RNasin; 0.5 U/μL SUPERase RNase inhibitor; 1 mM DTT; 0.15 mM spermine; 0.5 mM spermidine; 10 mM citric acid.

### 30% Iodixanol

30% OptiPrep in CitraHB; 0.5 U/μL RNasin; 0.5 U/μL SUPERase RNase inhibitor; 1 mM DTT; 0.15 mM spermine; 0.5 mM spermidine; 10 mM citric acid.

### 40% Iodixanol

40% OptiPrep in CitraHB; 0.5 U/μL RNasin; 0.5 U/μL SUPERase RNase inhibitor; 1 mM DTT; 0.15 mM spermine; 0.5 mM spermidine; 10 mM citric acid.

### OptiPrep™ (Stemcell Technologies, Catalog #07820)

Density gradient medium for the isolation and purification of viruses, macromolecules, a wide range of cells, as well as organelles such as nuclei, mitochondria, endosomes, or exosomes. According to the manufacturer, this flexible, versatile, and gentle density gradient medium is non-ionic, iodixanol-based (60% w/v), and has a density of 1.320±0.001 g/mL.

The chemical name for iodixanol is 5-[acetyl-[3-[acetyl-[3,5-bis(2,3-dihydroxypropylcarbamoyl)-2,4,6-triiodo-phenyl]amino]-2-hydroxy-propyl]amino]-N,N′-bis(2,3- dihydroxypropyl)-2,4,6-triiodo-benzene-1,3-dicarboxamide.

### Nuclei Wash

1×PBS, 1% BSA, 0.5 U/μL RNasin; 0.5 U/μL SUPERase RNase inhibitor.

### Example 2 Analysis and Characterization of Enteric Glial Cells

Using the nuclear RNA isolation method described in Example 1, this example provides results of analyzing enteric glial cells.

First, to determine whether enteric glia are analogous to any of the glial cell types in the central or peripheral nervous system, snRNA-seq was performed on mouse duodenal and cortical tissues (using nuclear RNA isolated using the CitraPrep method described in Example 1), in view of publicly available snRNA-seq data from all three nervous systems.

When ectodermal-derived glial cell types were computationally separated from all other cells, 7 glial cell types across 9 tissue regions were identified, extending throughout the body from the cortex (head) to the sural nerve (feet) (data not shown). After merging central and peripheral nervous system glial cells with enteric glia and controlling for batch effects, 19 transcriptionally unique clusters were identified through unsupervised clustering (data not shown). Importantly, clusters were not defined by sample or tissue origin, and nuclei from the same cell types but originating from separate studies overlapped in identity (data not shown).

As expected, expression of many individual transcripts was conserved in glia across the three nervous systems. Strikingly, however, enteric glia clustered mostly apart while closely related glial cell types like ependymal cells and tranycytes in the ventromedial hypothalamus mostly populated the same clusters (data not shown).

Enteric glia express many known glial cell genes, but in unusual combinations that conferred an identity distinct from other glia in the body. Analysis of enriched transcripts between cell types showed genetic programs related to known functions in defined glial cell types. Enteric glia were enriched for processes associated not only with known enteric glial functions such as maintenance of intestinal epithelium, purine metabolism, and smooth muscle contraction, but also with previously undescribed functions such as lipid absorption, cholesterol metabolism, and regulation of hormone levels.

Highly enriched, differentially expressed glial cell type specific markers were further identified and validated (data not shown). Interestingly, enteric glia express genes that are also expressed in the brain and have been linked to neurological diseases and disorders that harbor gut comorbidities of undefined origin. This includes Parkinson's disease (SCNA, LRRK2, and PRKN), amyotrophic lateral sclerosis 78 (C9ORF72, SOD1, TARDBP, and FUS), leukodystrophies (GFAP and PLP1), schizophrenia (TRIO, RB1CC1, XPO7, CUL1, HERC1, and GRIA3) and autism spectrum disorder (including but not limited to CHD2, SETBP1, KCNQ3, SOX5, DYRK1A, and TCF4, high confidence SFARI score 1/1S).

The data support that enteric glia are not a peripheral equivalent of astrocytes or other known glial cell types in the body, but instead are a molecularly distinct glial cell type.

Beyond their distinction from central nervous system glia, there is evidence of further diversity within enteric glia. For example, enteric glia elicit calcium responses to different stimuli specific to the muscle layer of the duodenum when compared to the colon. Hence, the following data demonstrates that enteric glial gene signatures diverge between the duodenum and the colon.

We merged our data sets containing enteric glial cells from the whole duodenum with two sources: enteric glial cells from sorted ENS cells from the myenteric plexus of the colon and from mechanically isolated colonic mucosa. Indeed, it was found that the majority of cells were separated by regional identity, and that the overall molecular profiles between duodenal and colonic enteric glia were distinct (data not shown).

Enteric glia outnumber neurons in the mammalian intestine but their subtypes are not well defined. Upon selection and re-clustering of ENS cells, 7 molecularly distinct enteric glia subpopulations were identified (data not shown). Analysis of these clusters revealed molecular blueprints of enteric glia that were previously not described. A cluster of enteric glia with enriched expression of the PPAR nuclear hormone receptors, which are key regulators of lipid metabolism and inflammation (data not shown). A single cluster had enriched expression of Sox6, Nrp1, Vim, Nfia, and other genes indicative of a less differentiated state while concurrently expressing transcripts of differentiated glial cells like Gfap. This transcriptionally poised state resembles that of pancreatic hub cells, a subpopulation of beta cells that transcriptionally display features of both mature and immature cells and act as pacemaker cells to synchronize release of insulin upon glucose stimulation. We named these cells enteric glial “hub cells”.

Enteric glia are known to secrete WNT ligands like WNT6 to promote regeneration of damaged intestinal epithelium. Consistently, high expression of Wnt6 was found specifically in EGC6, while Wnt4 and Wnt5a were highly expressed and specifically enriched in enteric glial hub cells (data not shown).

The entire enteric glia population was visualized through labeling the ENS and immunostaining of Sox10-cre; RCL-tdTomato mouse tissue, where tdTomato marks all neural crest derived cells. When we then performed immunostaining using markers of non-canonical enteric glial cell subpopulations identified through snRNAseq, we were able to discern the broader heterogeneity of enteric glial cells (data not shown).

These data provide a foundational overview of enteric glia populations in the adult gut and resolve the molecular profiles of enteric glial subtypes (data not shown). Four morphologically diverse enteric glial cells have been described, but it is not known whether these identities correspond to specific molecular subtypes.

Using an in vitro cell culture system optimized to grow primary enteric glial cells at scale, we demonstrated the presence of the four previously defined morphological enteric glia subtypes and identified two additional subpopulations call “triad” and “bipolar” in respect to cell shape (data not shown). The morphology of the molecularly defined enteric glial subpopulations were assessed, and the proportion of cells with different morphological identities in culture was quantitated by immunostaining.

Focusing on enteric glial hub cells, a striking enrichment of high NFIA expression, an enteric glial hub cell enriched marker, was found, with type 1 intraganglionic morphology (data not shown). We next conducted immunostaining of Sox 10-cre; RCL-tdTomato mouse tissue for markers of different enteric glia subtypes and detected widespread compartmentalization of enteric glial cells with some enriched in the submucosa, others in the mucosa, and a significant enrichment of glia expressing enteric glial hub cell markers in the muscle layer (data not shown). This finding places enteric glial hub cells in a location where they have the opportunity to impact peristalsis by interacting with neighboring neurons and muscle cells.

In summary, we show that the molecular profiles of enteric glial cells diverge in different spatial compartments. This transcriptional compartmentalization suggests that enteric glial cells in different layers of the intestine are functionally specialized.

Enteric glial hub cells are also predicted to interact with enteric neurons.

Equipped with 57,033 high-quality single nuclear transcriptomes from the entire adult duodenum, we mapped the connections between enteric glia and surrounding cells. Using receptor-ligand interactome analysis, we outlined predicted cell-cell interaction networks from each enteric glial subpopulation (data not shown). This analysis predicted that enteric glial hub cells interact with interstitial cells of Cajal (ICCs), telocytes, and enteric neurons (data not shown). As pancreatic hub cells act as specialized pacemaker cells to respond to glucose fluctuations in their environment and orchestrate islet oscillations, we hypothesized that enteric glial hub cells respond to signals to orchestrate function of cells in the muscle layer.

To determine how enteric glial hub cells become activated to potentially influence these specific cell types, we assessed expression of genes ontologically associated with monoatomic ion channel activity and filtered for the most highly significant subtype-enriched transcripts (data not shown). We noted significant expression of the mechanosensitive ion channel Piezo2 in enteric glial hub cells (data not shown). From these data, and given the spatial location of enteric glial hub cells, we hypothesized that enteric glial hub cells can influence the function of enteric neurons to regulate intestinal contractions and gut physiology through PIEZO2.

The data below demonstrates that the enteric glial hub cells act as biomechanical sensors to regulate intestinal motility.

Humans with PIEZO2 loss-of-function mutations report a wide range of bowel dysfunctions including constipation and hardened stools. It is known that PIEZO2 channels have a slight preference for Ca²⁺ ions; interestingly, activated enteric glia elicit dynamic Ca²⁺ responses. Genetic disruption of Ca²⁺ transients in Gfap+ enteric glia also leads to impaired gut motility, while forced influx of Ca²⁺ in Gfap+ enteric glia promotes gut motility. With this in mind, we set out to determine whether enteric glial hub cells directly sense force through the PIEZO2-Ca²⁺ axis to regulate gut motility.

We first confirmed transcript and protein expression of PIEZO2 in myenteric glial cells by its co-expression with SOX10 (marking all adult myenteric glia) and GFAP (marking a subset of myenteric glia including enteric glial hub cells) in both mouse and human tissue (data not shown). Every PIEZO2+ enteric glial cell that we observed co-expressed the enteric glia marker GFAP (data not shown), and Piezo2 transcript itself was highly enriched in enteric glial hub cells (data not shown); thus, targeting PIEZO2 via Sox10 (all myenteric glia) or Gfap (all PIEZO2+ glia) would selectively disrupt PIEZO2 in enteric glial hub cells.

We next wanted to determine if enteric glia were competent to respond to mechanical force. We engineered a microfluidic biochip to mechanically stimulate tissue while simultaneously performing high-resolution calcium imaging (data not shown). By selectively inducing expression of the genetically encoded calcium-sensitive indicator GCaMP6f in adult Sox10-creERt2 mice, we specifically recorded enteric glia activity in our intestine-on-a-chip platform. We generated ex vivo preparations of longitudinal muscle with the myenteric plexus by microdissection and visualized changes in enteric glia activity via GCaMP6f before and after 1 dyn/cm² shear stimulation. As expected, we observed differential calcium responses across enteric glia, with some cells spontaneously active without any stimuli. We also identified a subset of myenteric glial cells that were mechanosensitive, responding only after stimulation with shear stress. In the majority of mechanoresponsive enteric glia, activation was reversed upon treatment with D-GsMTx4, which inhibits PIEZO2 (data not shown). Immunostaining of these mechanosensory cells post-hoc showed that they expressed PIEZO2 and GFAP (data not shown). These results support that a specialized mechanosensory subpopulation of enteric glia exists in the muscle layer of the small intestine.

To determine whether PIEZO2 conferred functional specialization to mechanically sensitive enteric glial hub cells, we crossed Piezo2 fl/fl mice to the Sox10-creERt2 driver line. Treatment of mice with tamoxifen at three weeks of age allowed us to circumvent perinatal lethality of Piezo2 loss and, importantly, enabled us to selectively target myenteric glial cells in the gut as they are the only cells in the adult gastrointestinal tract that express Sox10 (54). Three weeks after tamoxifen administration, we confirmed Piezo2 deletion by immunostaining, which showed loss of PIEZO2 from SOX10+ enteric glia in the adult mouse duodenum (data not shown).

To understand how PIEZO2 loss in enteric glia affects gastrointestinal functions, we gavaged mice with a non-absorbable dye and measured the length of time for the colored fecal pellet to appear (data not shown). In mice lacking PIEZO2 in all myenteric glia in the gut, we observed a significant delay in overall transit time compared to co-housed, littermate controls (data not shown), along with a decrease in fecal water content likely due to slower gut movement (data not shown). PIEZO2 deficient mice did not exhibit any differences in epithelial permeability, weight, or intestinal length compared to control littermates (data not shown).

To identify the gastrointestinal segment where luminal transit was delayed, we gavaged mice with a non-absorbable fluorescent dye and collected the luminal contents from equally sized segments of the gut after 30 minutes (data not shown). We found a delay in transit early in the gastrointestinal tract of Piezo2 fl/fl; Sox10-creERt2 mice compared to littermate controls; most notably we observed a significant defect in gastric emptying demonstrated by more dye sequestered in the stomach (data not shown). The defects observed after loss of PIEZO2 from enteric glia resemble a condition where gastric emptying is delayed in individuals called gastroparesis, a common comorbidity in neurological diseases.

Harnessing the intestine-on-a-chip platform, we performed ex vivo contractility assays to assess neuron and muscle activity in tissue after loss of PIEZO2. In response to shear stress, we observed a regular oscillating contractile pattern that was completely abolished upon treatment with the voltage-dependent L-type calcium channel inhibitor nifedipine (data not shown). In contrast, while Piezo2 fl/fl; Sox 10-creERt2 tissue had comparable amplitude or strength of contractions, these tissues exhibited arrhythmicity compared to littermate controls (data not shown). The wave-like, rhythmic movements of the intestine called peristalsis are at least partially controlled through release of acetylcholine and nitric oxide by enteric neurons. Acetylcholine promotes contraction while nitric oxide induces relaxation of muscle; this relay of signaling must be tightly controlled for regulated, unidirectional movement of contents through the gastrointestinal tract. As enteric glial hub cells are predicted to interact with enteric neurons (data not shown), and loss of Piezo2 from enteric glia leads to defects in peristalsis, we analyzed the levels of acetylcholine and nitric oxide in the muscle layer of the small intestine in Piezo2 fl/fl; Sox10-creERt2. Indeed, neurotransmission in the myenteric plexus was disrupted, with a decrease in levels of nitric oxide and elevated levels of acetylcholine (data not shown).

Taken together, these data show that enteric glia regulate gut physiology by fine-tuning neurotransmission to control the oscillatory contractions in muscle tissue. Expression of Piezo2 is highly enriched in enteric glial hub cells while Sox10 drives PIEZO2 deletion in all adult myenteric glia. While many enteric glial cells co-express glial markers SOX10, GFAP, and PLP1, subsets of enteric glia differentially expressed these markers. Our analyses show that all PIEZO2+ enteric glia co-express GFAP (data not shown). Computational selection of Plp1+ and Gfap+ cells revealed at the transcriptional level higher expression of Piezo2 in cells co-expressing Gfap compared to Plp1 (data not shown).

With this in mind, we used genetic drivers Plp1-creERt2 and Gfap-creERt2 crossed with Piezo2 fl/fl to specifically delete Piezo2 in subsets of adult enteric glial cells. In mice with loss of PIEZO2 from Gfap+ cells, we detected a significant delay in transit time, and significant retention of dye in the stomach, phenocopying the global myenteric glia knockout of PIEZO2 (data not shown). In contrast, Plp1-driven loss of PIEZO2 had no phenotypic difference from littermate controls (data not shown). These data show that enteric glial hub cells are a functionally specialized subtype of enteric glia that express PIEZO2 and fine-tune intestinal physiology.

1. A method for isolating nuclear RNA from a high degradation tissue, said method comprising:
i) incubating on ice the high degradation tissue reduced to a suitable size in a lysis buffer, for about 5-9 minutes (e.g., about 6-8 minutes, about 6.5-7.5 minutes, or about 7 minutes), wherein the lysis buffer comprises RNase inhibitor, protease inhibitor, buffer, osmotic stabilizer and ions to maintain/stabilize organelle membrane integrity, DNA precipitation reagent, chelating agent that quenches free Mg²⁺, and/or optionally anti-oxidant or reducing agent;
ii) homogenizing the high degradation tissue of the suitable size in the lysis buffer through sheer mechanical stress to generate a loosely homogenized tissue, wherein the sheer mechanical stress is generated by a Dounce-type homogenizer having a loose or Type-A pestle, through a total of about 15-25 passes (e.g., about 20 passes) of the Type-A pestle in the Dounce-type homogenizer, wherein the outer diameter of the Type-A homogenizer has a clearance from the inner diameter of the cylinder wall of between about 0.0025-0.0055 inches;
iii) adding a detergent mix, at about 6-10% (e.g., about 8%) volume of the lysis buffer, to the loosely homogenized tissue, wherein the detergent mix comprises about 4-6% or about 5% of IGEPAL CA-630-type nonionic, non-denaturing detergent in the lysis buffer;
iv) homogenizing the loosely homogenized tissue with the detergent mix in order to release organelles into said lysis buffer, using a tight or Type-B pestle in the Dounce-type homogenizer, by about 35-45 passes (e.g., about 40 passes) of the Type-B pestle in the Dounce-type homogenizer, wherein the outer diameter of the Type-B homogenizer has a clearance from the inner diameter of the cylinder wall of between about 0.0005-0.0025 inches;
v) filtering tissue lysates generated by step iv) through a strainer having a sieve size of about 40 μm to collect nuclei in the pass-through portion;
vi) adding an equal volume of a 50% iodixanol solution to the pass-through portion and vortexing to create a mixture having about 25% iodixanol;
vii) underlaying the mixture with an about 30% iodixanol solution and an about 40% iodixanol solution without disturbing the resulting three-layer iodixanol gradient;
viii) centrifuging the three-layer iodixanol gradient at about 10,000 g for 15-25 minutes (e.g., about 18 minutes) at about 4° C. with no brake;
ix) collecting nuclei from the interface(s) between the layers of the iodixanol gradient into a nuclei wash buffer for washing through vortexing, up to twice; and,
x) filtering washed nuclei through a 20 μm filter before pelleting the nuclei by centrifugation at about 500 g (e.g., for about 5 minutes at about 4° C.).

2. The method of claim 1, wherein the high degradation tissue derives from a surgical resection or autopsy, or is rich in RNAses, proteases, enzymes, and/or ions (e.g., an enteric tissue, such as the most proximal region of the small intestine, duodenum, ileum, colon, or pancreas).

3. The method of claim 1 or 2, wherein the suitable size is no more than about 3 mm, 2 mm, or 1 mm in any dimension.

4. The method of any one of claims 1-3, wherein step i) is about 7 minutes on ice, optionally with about 4 mL of the lysis buffer for a total tissue amount of less than about 15 mm in any dimension.

5. The method of any one of claims 1-4, wherein the lysis buffer comprises:
a) an RNase inhibitor selected from the group consisting of RNasin (e.g., about 0.25-1 U/μL, or about 0.5 U/μL of RNasin) and SUPERase RNAse Inhibitor (e.g., about 0.25-1 U/μL, or about 0.5 U/μL of SUPERase RNAse Inhibitor);
b) a protease inhibitor (e.g., complete, EDTA-free protease inhibitor);
c) a buffer that maintains pH at about 7.5-8 (e.g., about 20 mM Tricine-KOH, pH 7.8);
d) a DNA precipitation reagent selected from the group consisting of spermine tetrahydrochloride (e.g., about 0.15 mM spermine tetrahydrochloride) and spermidine trihydrochloride (e.g., about 0.5 mM spermidine trihydrochloride);
e) an osmotic stabilizer (such as 0.25 M sucrose);
f) ions to maintain/stabilize organelle membrane integrity (such as about 20-30 mM or about 25 mM KCl, and about 2.5-7.5 mM or about 5 mM MgCl 2);
g) a chelating agent to quench free Mg²⁺ (such as about 5-20 mM or about 10 mM citric acid); and,
h) an anti-oxidant or reducing agent (such as about 1 mM DTT).

6. The method of any one of claims 1-5, wherein the IGEPAL CA-630-type nonionic, non-denaturing detergent comprises IGEPAL CA-630 (octylphenoxypolyethoxyethanol) or Nonidet P-40 (NP-40).

7. The method of any one of claims 1-6, wherein the concentrations of the components of the lysis buffer remain substantially unchanged through steps i)-viii).

8. The method of any one of claims 1-7, wherein step ix) is performed in about 10 ml of the nuclei wash buffer.

9. The method of any one of claims 1-8, wherein step ix) consists of two washes, each in about 10 mL of the nuclei wash buffer.

10. The method of any one of claims 1-9, further comprising resuspending collected nuclei in the nuclei wash buffer for counting (e.g., in hemocytometer) and/or observation for quality control.

11. The method of any one of claims 1-10, wherein said nuclei wash buffer comprises 1% serum albumin (such as BSA), an RNase inhibitor (such as RNasin (e.g., about 0.25-1 U/μL, or about 0.5 U/μL of RNasin) and/or SUPERase RNAse Inhibitor (e.g., about 0.25-1 U/μL, or about 0.5 U/μL of SUPERase RNAse Inhibitor)) in a buffer (such as PBS).

12. The method of any one of claims 1-10, wherein nuclear RNA isolated by the method has an 28S RNA:18S RNA ratio of at least about 50, 55, 60, 65, 70, 75, 80, 85 or more.

13. A method of profiling transcriptome based on single nucleus RNA-sequencing (snRNA-seq), the method comprising:
1) isolating nuclear RNA using the method of any one of claims 1-12; and,
2) profiling transcriptome of the isolated nuclear RNA using single nucleus RNA-sequencing (snRNA-seq).

14. The method of claim 13, wherein the nuclear RNA is isolated from a high degradation tissue that either derives from a surgical resection or autopsy, or is rich in RNAses, proteases, enzymes, and/or ions (e.g., an enteric tissue, such as the most proximal region of the small intestine, duodenum, ileum, colon, or pancreas).
